(19) United States
(12) Patent Application Publication
(10) Pub. No.: US 2023/0233698 A1
(43) Pub. Date: Jul. 27, 2023

(54) TREATMENT OF DISEASES CHARACTERIZED BY OVEREXPRESSION OF ERYTHROPOIETIN-PRODUCING HEPATOCELLULAR RECEPTOR A2 (EPHA2)

(71) Applicant: BicycleTx Limited, Cambridge (GB)

(72) Inventors: Gavin BENNETT, Cambridge (GB); Stephen BLAKEMORE, Littleton, MA (US); Carly CAMPBELL, Arlington, MA (US); Michael RIGBY, Cambridge (GB)

(21) Appl. No.: 18/001,374

(22) PCT Filed: Jun. 11, 2021
(86) PCT No.: PCT/GB2021/051451

§ 371 Date: Dec. 9, 2022

Related U.S. Application Data
(60) Provisional application No. 63/038,279, filed on Jun. 12, 2020.

(51) Int. Cl. A61K 47/64; C07K 16/28; A61K 47/65; A61P 35/00; G01N 33/574

(52) U.S. Cl. CPC A61K 47/6415; C07K 16/2866; A61K 47/65; A61P 35/00; G01N 33/57484

(57) ABSTRACT

The present invention relates to a Bicycle toxin conjugate BT5528, or pharmaceutically acceptable salts thereof, or pharmaceutical compositions thereof, and uses thereof.

### TECHNICAL FIELD OF THE INVENTION

The present invention relates to Bicycle toxin conjugates specific for EphA2, or pharmaceutically acceptable salts thereof, or pharmaceutical compositions thereof, and uses for preventing or treating a disease, disorder, or condition characterized by overexpression of Erythropoietin-producing hepatocellular receptor A2 (EphA2) in a diseased tissue, for example, in a tumor tissue.

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å²; Wu et al. (2007), Science 330, 1066-71), a cyclic peptide with the Arg-Gly-Asp motif binding to integrin αVb3 (355 Å²) (Xiong et al. (2002), Science 296 (5565), 151-5) or the cyclic peptide inhibitor upain-1 binding to urokinase-type plasminogen activator (603 Å²; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8, (MMP-8) which lost its selectivity over other MMPs when its ring was opened (Cherney et al. (1998), J Med Chem 41 (11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example TATA (1,1′,1″-(1,3,5-triazinane-1,3,5-triyl)triprop-2-en-1-one, Heinis et al. Angew Chem, Int Ed. 2014; 53:1602-1606).

Phage display-based combinatorial approaches have been developed to generate and screen large libraries of bicyclic peptides to targets of interest (Heinis et al. (2009), Nat Chem Biol 5 (7), 502-7 and WO 2009/098450). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule scaffold.

### SUMMARY OF THE INVENTION

As described herein, the inventors have discovered that levels of EphA2 in a diseased tissue are indicative of patient responsiveness to treatment with a Bicycle toxin conjugate specific for EphA2. EphA2 is overexpressed in many difficult to treat tumors, such as NSCLC, TNBC, pancreatic, ovarian, gastric/upper GI, and urothelial cancers. EphA2 is expressed at relatively low levels in normal adult tissues. EphA2 has been targeted by certain other drugs, which failed in the clinic due to unacceptable toxicity.

In one aspect, the invention provides a method of identifying or selecting a patient having an elevated EphA2 level in a diseased tissue, comprising measuring EphA2 level in a diseased tissue of a patient, and selecting a patient having an elevated EphA2 level in the diseased tissue.

In another aspect, provided herein is a method of treating a disease in a patient having an elevated EphA2 level in a diseased tissue, for example, as determined using a method described herein, comprising administering to the patient a Bicycle toxin conjugate specific for EphA2, or a pharmaceutically acceptable salt thereof, or a pharmaceutical composition thereof.

In another aspect, the present invention provides a method of treating a disease in a patient, comprising selecting a patient having an elevated EphA2 level in a diseased tissue, for example, using a method as described herein, and administering to the patient a Bicycle toxin conjugate specific for EphA2, or a pharmaceutically acceptable salt thereof, or a pharmaceutical composition thereof.

In some embodiments, a disease is a cancer, for example, the cancer as described herein. In some embodiments, a diseased tissue is a tumor tissue. In some embodiments, a Bicycle toxin conjugate specific for EphA2 is selected from those as described herein.

### DETAILED DESCRIPTION OF CERTAIN EMBODIMENTS

### 1. General Description of Certain Embodiments of the Invention

The EphA2 levels in tumor tissues have been measured by IHC staining assays. It has been found that the EphA2 levels on tumor cell membrane and in tumor cell cytoplasm are indicative of tumor responsiveness to treatment with a Bicycle toxin conjugate specific for EphA2. Without wishing to be bound by any particular theory or mechanism, the inventors have discovered that a tumor having an elevated EphA2 level in a diseased tissue is more likely to benefit from a treatment with a Bicycle toxin conjugate specific for EphA2. It has also been found that a tumor having an elevated EphA2 level on tumor cell membrane is more likely to benefit from a treatment with BT5528.

Accordingly, in one aspect, the invention provides a method of identifying or selecting a patient having an elevated EphA2 level in a diseased tissue, comprising measuring EphA2 level in a diseased tissue of a patient, and selecting a patient having an elevated EphA2 level in the diseased tissue.

In another aspect, provided herein is a method of treating a disease in a patient having an elevated EphA2 level in a diseased tissue, for example, as determined using a method described herein, comprising administering to the patient a Bicycle toxin conjugate specific for EphA2, or a pharmaceutically acceptable salt thereof, or a pharmaceutical composition thereof.

In another aspect, the present invention provides a method of treating a disease in a patient, comprising selecting a patient having an elevated EphA2 level in a diseased tissue, for example, using a method as described herein, and administering to the patient a Bicycle toxin conjugate specific for EphA2, or a pharmaceutically acceptable salt thereof, or a pharmaceutical composition thereof.

### 2. Compounds and Definitions

As used herein, the term “a Bicycle toxin conjugate specific for EphA2” refers to a Bicycle toxin conjugate that binds specifically to EphA2. Various Bicycle toxin conjugates specific for EphA2 have been described previously, for example, in US 2019/0184025, WO 2019/122861, and WO 2019/122863, the content of each of which is incorporated herein by reference in its entirety.

The term “BT5528,” as used herein, is a Bicycle toxin conjugate having a structure as shown below, or a pharmaceutically acceptable salt thereof, wherein the molecular scaffold is 1,1′,1″-(1,3,5-triazinane-1,3,5-triyl)triprop-2-en-1-one (TATA), and the peptide ligand comprises the amino acid sequence:

(β-Ala)-Sar₁₀-A(HArg)D-Cᵢ(HyP)LVNPLCᵢᵢLHP(D-Asp)W(HArg)Cᵢᵢᵢ (SEQ ID NO: 1) wherein Sar is sarcosine, HArg is homoarginine, and HyP is hydroxyproline.

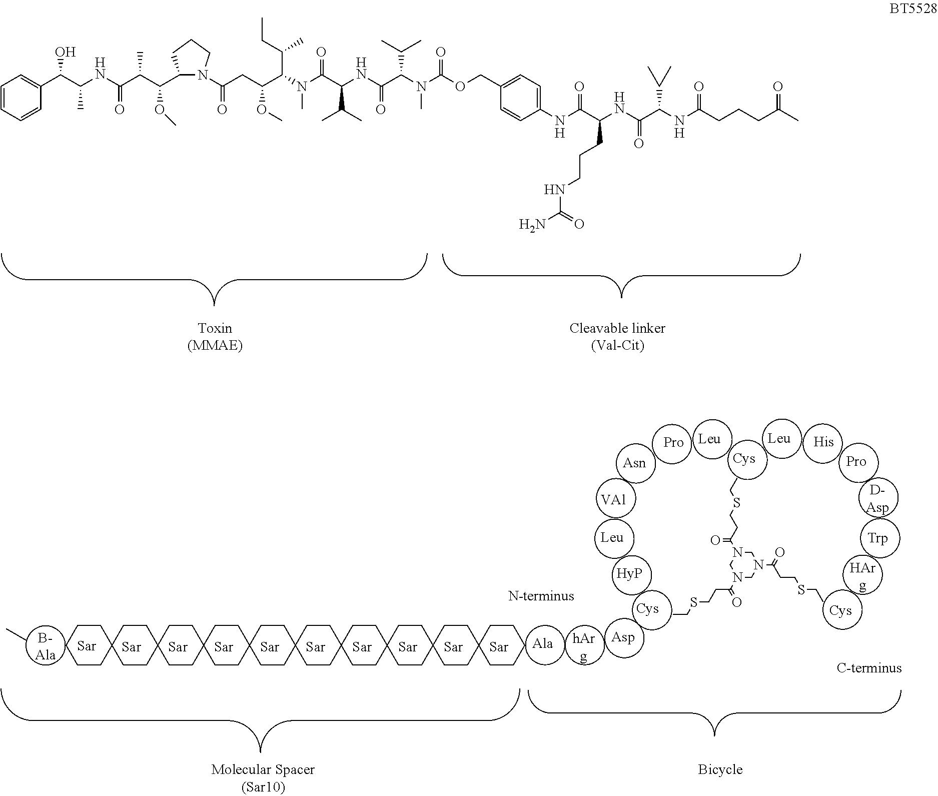

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

As used herein, the terms “about” or “approximately” have the meaning of within 20% of a given value or range. In some embodiments, the term “about” refers to within 20%, 19%, 18%, 17%, 16%, 15%, 14%, 13%, 12%, 11%, 10%, 9%, 8%, 7%, 6%, 5%, 4%, 3%, 2%, or 1% of a given value.

### 3. Description of Exemplary Embodiments of the Invention

In some embodiments, the invention provides a method of identifying or selecting a patient having an elevated EphA2 level in a tumor tissue, comprising measuring EphA2 level in a tumor tissue of a patient, and selecting a patient having an elevated EphA2 level in the tumor tissue. In some embodiments, the method further comprises administering a Bicycle toxin conjugate specific for EphA2, or a pharmaceutically acceptable salt thereof, or a pharmaceutical composition thereof, to a patient having an elevated EphA2 level in a tumor tissue.

In some embodiments, a patient is a patient having pancreatic cancer. In some embodiments, a patient is a patient having stomach cancer. In some embodiments, a patient is a patient having bladder cancer. In some embodiments, a patient is a patient having head & neck cancer. In some embodiments, a patient is a patient having non-small cell lung cancer (NSCLC). In some embodiments, a patient is a patient having triple negative breast cancer (TNBC). In some embodiments, a patient is a patient having ovarian cancer.

In some embodiments, a tumor tissue is a pancreatic tumor tissue. In some embodiments, a tumor tissue is a stomach tumor tissue. In some embodiments, a tumor tissue is a bladder tumor tissue. In some embodiments, a tumor tissue is a head & neck tumor tissue. In some embodiments, a tumor tissue is a non-small cell lung cancer (NSCLC) tumor tissue. In some embodiments, a tumor tissue is a triple negative breast cancer (TNBC) tumor tissue. In some embodiments, a tumor tissue is an ovarian tumor tissue.

As used herein, the term “an elevated EphA2 level” refers to that certain percentage of cells in a tumor tissue have a detectable amount of EphA2, for example, on tumor cell membrane, or in tumor cell cytoplasm, or both. In some embodiments, EphA2 positive refers to that about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of cells in a tumor tissue have a detectable amount of EphA2, for example, on tumor cell membrane, or in tumor cell cytoplasm, or both.

There are a variety of methods to measure the amount of EphA2 in a tissue. In some embodiments, a method of measuring EphA2 level in a tumor tissue of a patient comprises using an EphA2 immunohistochemistry (IHC) staining assay. In some embodiments, an EphA2 IHC staining assay comprises staining a tumor tissue section using a human EphA2 antibody. In some embodiments, a human EphA2 antibody selectively binds to the extracellular domain (ECD) of EphA2. In some embodiments, a human EphA2 antibody selectively binding to the ECD of EphA2 is human EphA2 antibody AF3035. In some embodiments, a human EphA2 antibody selectively binds to the cytoplasmic domain of EphA2. In some embodiments, a human EphA2 antibody selectively binding to the cytoplasmic domain of EphA2 is human EphA2 antibody CST6997.

In some embodiments, a human EphA2 antibody is at a concentration of up to about 50 μg/mL. In some embodiments, a human EphA2 antibody is at a concentration of up to about 40 μg/mL. In some embodiments, a human EphA2 antibody is at a concentration of up to about 30 μg/mL. In some embodiments, a human EphA2 antibody is at a concentration of up to about 20 μg/mL. In some embodiments, a human EphA2 antibody is at a concentration of up to about 10 μg/mL. In some embodiments, a human EphA2 antibody is at a concentration of about 5 μg/mL, about 6 μg/mL, about 7 μg/mL, about 8 μg/mL, about 9 μg/mL, about 10 μg/mL, about 11 μg/mL, about 12 μg/mL, about 13 μg/mL, about 14 μg/mL, or about 15 μg/mL. In some embodiments, a human EphA2 antibody selectively binding to the ECD of EphA2, such as AF3035, is at a concentration of about 5 μg/mL, about 6 μg/mL, about 7 μg/mL, about 8 μg/mL, about 9 μg/mL, about 10 μg/mL, about 11 μg/mL, about 12 μg/mL, about 13 μg/mL, about 14 μg/mL, or about 15 μg/mL. In some embodiments, a human EphA2 antibody selectively binding to the ECD of EphA2, such as AF3035, is at a concentration of about 10 μg/mL.

In some embodiments, the invention provides a method of identifying or selecting a patient having an elevated EphA2 level in a tumor tissue, comprising measuring staining intensity in a tumor tissue section of a patient using an EphA2 IHC staining assay, and selecting a patient who is staining positive in the EphA2 IHC staining assay. In some embodiments, an EphA2 IHC staining assay is as described in Example 2 herein.

As used herein, the term “a patient who is staining positive” refers to a patient having certain percentage of cells in a tumor tissue section which are staining positive in an EphA2 IHC staining assay. In some embodiments, a patient who is staining positive has about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of cells in a tumor tissue section which are staining positive in an EphA2 IHC staining assay.

There are a variety of methods to measure staining intensity in an IHC staining assay. In some embodiments, staining intensity is measured by visual scoring, for example, by manual scoring using conventional light microscopy. In some embodiments, staining intensity is measured by computational tissue analysis (CTA) scoring. The staining intensity levels can be no staining (0), weak staining (1+), median staining (2+), or strong staining (3+). In some embodiments, staining intensity is measured on tumor cell membrane of a tumor tissue section. In some embodiments, staining intensity is measured in tumor cell cytoplasm of a tumor tissue section. In some embodiments, staining intensity is measured both on tumor cell membrane and in tumor cell cytoplasm of a tumor tissue section.

In some embodiments, staining positive refers to an H-score of about 15 or more in a tumor tissue section in an IHC staining assay. In some embodiments, staining positive refers to an H-score of about 20 or more in a tumor tissue section in an IHC staining assay. In some embodiments, staining positive refers to an H-score of about 30 or more in a tumor tissue section in an IHC staining assay. In some embodiments, staining positive refers to an H-score of about 40 or more in a tumor tissue section in an IHC staining assay. In some embodiments, staining positive refers to an H-score of about 50 or more in a tumor tissue section in an IHC staining assay. In some embodiments, staining positive refers to an H-score of about 75 or more in a tumor tissue section in an IHC staining assay. In some embodiments, staining positive refers to an H-score of about 100 or more in a tumor tissue section in an IHC staining assay. In some embodiments, staining positive refers to an H-score of about 125 or more in a tumor tissue section in an IHC staining assay. In some embodiments, staining positive refers to an H-score of about 150 or more in a tumor tissue section in an IHC staining assay.

An H-score is the sum of the products of the percent of cells x their staining intensity on a scale of 0-3 as described above (no staining (0), weak staining (1+), median staining (2+), or strong staining (3+)):

[((0×(% cells at 0))+((1×(% cells at 1+))+((2×(% cells at 2+))+((3×(% cells at 3))]

An H-score can be generated for different compartment in a tumor tissue section, including, for example, the tumor cell membrane and cytoplasm. In some embodiments, an H-score refers to an H-score for tumor cell membrane, which is the sum of the products of the percent of cells x their cell membrane staining intensity on a scale of 0-3 as described above. In some embodiments, an H-score refers to an H-score for tumor cell cytoplasm, which is the sum of the products of the percent of cells x their cytoplasm staining intensity on a scale of 0-3 as described above.

In some embodiments, staining positive refers to an H-score for tumor cell membrane of about 15 or more in a tumor tissue section in an IHC staining assay. In some embodiments, staining positive refers to an H-score for tumor cell membrane of about 20 or more in a tumor tissue section in an IHC staining assay. In some embodiments, staining positive refers to an H-score for tumor cell membrane of about 30 or more in a tumor tissue section in an IHC staining assay. In some embodiments, staining positive refers to an H-score for tumor cell membrane of about 40 or more in a tumor tissue section in an IHC staining assay. In some embodiments, staining positive refers to an H-score for tumor cell membrane of about 50 or more in a tumor tissue section in an IHC staining assay. In some embodiments, staining positive refers to an H-score for tumor cell membrane of about 75 or more in a tumor tissue section in an IHC staining assay. In some embodiments, staining positive refers to an H-score for tumor cell membrane of about 100 or more in a tumor tissue section in an IHC staining assay. In some embodiments, staining positive refers to an H-score for tumor cell membrane of about 125 or more in a tumor tissue section in an IHC staining assay. In some embodiments, staining positive refers to an H-score for tumor cell membrane of about 150 or more in a tumor tissue section in an IHC staining assay.

In some embodiments, staining positive refers to an H-score for tumor cell cytoplasm of about 15 or more in a tumor tissue section in an IHC staining assay. In some embodiments, staining positive refers to an H-score for tumor cell cytoplasm of about 20 or more in a tumor tissue section in an IHC staining assay. In some embodiments, staining positive refers to an H-score for tumor cell cytoplasm of about 30 or more in a tumor tissue section in an IHC staining assay. In some embodiments, staining positive refers to an H-score for tumor cell cytoplasm of about 40 or more in a tumor tissue section in an IHC staining assay. In some embodiments, staining positive refers to an H-score for tumor cell cytoplasm of about 50 or more in a tumor tissue section in an IHC staining assay. In some embodiments, staining positive refers to an H-score for tumor cell cytoplasm of about 75 or more in a tumor tissue section in an IHC staining assay. In some embodiments, staining positive refers to an H-score for tumor cell cytoplasm of about 100 or more in a tumor tissue section in an IHC staining assay. In some embodiments, staining positive refers to an H-score for tumor cell cytoplasm of about 125 or more in a tumor tissue section in an IHC staining assay. In some embodiments, staining positive refers to an H-score for tumor cell cytoplasm of about 150 or more in a tumor tissue section in an IHC staining assay.

In some embodiments, the invention provides a method of identifying or selecting a patient having an elevated EphA2 level in a tumor tissue, comprising measuring staining intensity in a tumor tissue section of a patient using an EphA2 IHC staining assay, and selecting a patient having an H-score of about 15 or more, about 20 or more, about 30 or more, about 40 or more, about 50 or more, about 75 or more, about 100 or more, about 125 or more, or about 150 or more.

In some embodiments, the invention provides a method of identifying or selecting a patient having an elevated EphA2 level in a tumor tissue, comprising measuring staining intensity in a tumor tissue section of a patient using an EphA2 IHC staining assay, and selecting a patient having an H-score for tumor cell membrane of about 15 or more, about 20 or more, about 30 or more, about 40 or more, about 50 or more, about 75 or more, about 100 or more, about 125 or more, or about 150 or more.

In some embodiments, the invention provides a method of identifying or selecting a patient having an elevated EphA2 level in a tumor tissue, comprising measuring staining intensity in a tumor tissue section of a patient using an EphA2 IHC staining assay, and selecting a patient having an H-score for tumor cell cytoplasm of about 15 or more, about 20 or more, about 30 or more, about 40 or more, about 50 or more, about 75 or more, about 100 or more, about 125 or more, or about 150 or more.

In some embodiments, the present invention provides a method of treating a cancer in a patient having an elevated EphA2 level in a tumor tissue, comprising administering to the patient a Bicycle toxin conjugate specific for EphA2, or a pharmaceutically acceptable salt thereof, or a pharmaceutical composition thereof. In some embodiments, an elevated EphA2 level is as described herein.

In some embodiments, the present invention provides a method of treating a cancer in a patient, comprising selecting a patient having an elevated EphA2 level in a tumor tissue, and administering to the patient a Bicycle toxin conjugate specific for EphA2, or a pharmaceutically acceptable salt thereof, or a pharmaceutical composition thereof. In some embodiments, an elevated EphA2 level is as described herein.

In some embodiments, the present invention provides a method of treating a cancer in a patient, comprising measuring EphA2 level in a tumor tissue section of a patient, selecting a patient having an elevated EphA2 level in a tumor tissue, and administering to the patient a Bicycle toxin conjugate specific for EphA2, or a pharmaceutically acceptable salt thereof, or a pharmaceutical composition thereof. In some embodiments, an elevated EphA2 level is as described herein.

In some embodiments, a cancer is pancreatic cancer. In some embodiments, a cancer is stomach cancer. In some embodiments, a cancer is bladder cancer. In some embodiments, a cancer is head & neck cancer. In some embodiments, a cancer is non-small cell lung cancer (NSCLC). In some embodiments, a cancer is a triple negative breast cancer (TNBC). In some embodiments, a cancer is ovarian cancer.

In some embodiments, the present invention provides a method of treating a cancer in a patient having an H-score of about 15 or more, about 20 or more, about 30 or more, about 40 or more, about 50 or more, about 75 or more, about 100 or more, about 125 or more, or about 150 or more in a tumor tissue section in an EphA2 IHC staining assay, comprising administering to the patient a Bicycle toxin conjugate specific for EphA2, or a pharmaceutically acceptable salt thereof, or a pharmaceutical composition thereof. In some embodiments, an EphA2 IHC staining assay is as described herein.

In some embodiments, the present invention provides a method of treating a cancer in a patient having an H-score for tumor cell membrane of about 15 or more, about 20 or more, about 30 or more, about 40 or more, about 50 or more, about 75 or more, about 100 or more, about 125 or more, or about 150 or more in a tumor tissue section in an EphA2 IHC staining assay, comprising administering to the patient a Bicycle toxin conjugate specific for EphA2, or a pharmaceutically acceptable salt thereof, or a pharmaceutical composition thereof.

In some embodiments, the present invention provides a method of treating a cancer in a patient having an H-score for tumor cell cytoplasm of about 15 or more, about 20 or more, about 30 or more, about 40 or more, about 50 or more, about 75 or more, about 100 or more, about 125 or more, or about 150 or more in a tumor tissue section in an EphA2 IHC staining assay, comprising administering to the patient a Bicycle toxin conjugate specific for EphA2, or a pharmaceutically acceptable salt thereof, or a pharmaceutical composition thereof.

In some embodiments, the present invention provides a method of treating a cancer in a patient, comprising selecting a patient having an H-score of about 15 or more, about 20 or more, about 30 or more, about 40 or more, about 50 or more, about 75 or more, about 100 or more, about 125 or more, or about 150 or more in a tumor tissue section in an EphA2 IHC staining assay, and administering to the patient a Bicycle toxin conjugate specific for EphA2, or a pharmaceutically acceptable salt thereof, or a pharmaceutical composition thereof.

In some embodiments, the present invention provides a method of treating a cancer in a patient, comprising selecting a patient having an H-score for tumor cell membrane of about 15 or more, about 20 or more, about 30 or more, about 40 or more, about 50 or more, about 75 or more, about 100 or more, about 125 or more, or about 150 or more in a tumor tissue section in an EphA2 IHC staining assay, and administering to the patient a Bicycle toxin conjugate specific for EphA2, or a pharmaceutically acceptable salt thereof, or a pharmaceutical composition thereof.

In some embodiments, the present invention provides a method of treating a cancer in a patient, comprising selecting a patient having an H-score for tumor cell cytoplasm of about 15 or more, about 20 or more, about 30 or more, about 40 or more, about 50 or more, about 75 or more, about 100 or more, about 125 or more, or about 150 or more in a tumor tissue section in an EphA2 IHC staining assay, and administering to the patient a Bicycle toxin conjugate specific for EphA2, or a pharmaceutically acceptable salt thereof, or a pharmaceutical composition thereof.

In some embodiments, the present invention provides a method of treating a cancer in a patient, comprising measuring staining intensity in a tumor tissue section of a patient using an EphA2 IHC staining assay, selecting a patient having an H-score of about 15 or more, about 20 or more, about 30 or more, about 40 or more, about 50 or more, about 75 or more, about 100 or more, about 125 or more, or about 150 or more, and administering to the patient a Bicycle toxin conjugate specific for EphA2, or a pharmaceutically acceptable salt thereof, or a pharmaceutical composition thereof.

In some embodiments, the present invention provides a method of treating a cancer in a patient, comprising measuring staining intensity in a tumor tissue section of a patient using an EphA2 IHC staining assay, selecting a patient having an H-score for tumor cell membrane of about 15 or more, about 20 or more, about 30 or more, about 40 or more, about 50 or more, about 75 or more, about 100 or more, about 125 or more, or about 150 or more, and administering to the patient a Bicycle toxin conjugate specific for EphA2, or a pharmaceutically acceptable salt thereof, or a pharmaceutical composition thereof.

In some embodiments, the present invention provides a method of treating a cancer in a patient, comprising measuring staining intensity in a tumor tissue section of a patient using an EphA2 IHC staining assay, selecting a patient having an H-score for tumor cell cytoplasm of about 15 or more, about 20 or more, about 30 or more, about 40 or more, about 50 or more, about 75 or more, about 100 or more, about 125 or more, or about 150 or more, and administering to the patient a Bicycle toxin conjugate specific for EphA2, or a pharmaceutically acceptable salt thereof, or a pharmaceutical composition thereof.

In some embodiments, a Bicycle toxin conjugate specific for EphA2 is selected from the compounds as described in US 2019/0184025, WO 2019/122861, and WO 2019/122863, each of which is incorporated herein by reference in its entirety.

In some embodiments, a Bicycle toxin conjugate specific for EphA2 is BT5528 as described herein, or a pharmaceutically acceptable salt thereof.

In some embodiments, the present invention provides a method of treating a cancer in a patient having an elevated EphA2 level in a tumor tissue, comprising administering BT5528 to the patient, or a pharmaceutically acceptable salt thereof, or a pharmaceutical composition thereof.

In some embodiments, the present invention provides a method of treating a cancer in a patient having an H-score for tumor cell membrane of about 15 or more, about 20 or more, about 30 or more, about 40 or more, about 50 or more, about 75 or more, about 100 or more, about 125 or more, or about 150 or more in a tumor tissue section in an EphA2 IHC staining assay, comprising administering BT5528 to the patient, or a pharmaceutically acceptable salt thereof, or a pharmaceutical composition thereof.

In some embodiments, the present invention provides a method of treating a cancer in a patient, comprising selecting a patient having an elevated EphA2 level in a tumor tissue, and administering BT5528 to the patient, or a pharmaceutically acceptable salt thereof, or a pharmaceutical composition thereof.

In some embodiments, the present invention provides a method of treating a cancer in a patient, comprising selecting a patient having an H-score for tumor cell membrane of about 15 or more, about 20 or more, about 30 or more, about 40 or more, about 50 or more, about 75 or more, about 100 or more, about 125 or more, or about 150 or more, in a tumor tissue section in an IHC staining assay, and administering BT5528 to the patient, or a pharmaceutically acceptable salt thereof, or a pharmaceutical composition thereof.

In some embodiments, the present invention provides a method of treating a cancer in a patient, comprising measuring EphA2 level in a tumor tissue of a patient, selecting a patient having an elevated EphA2 level in a tumor tissue, and administering BT5528 to the patient, or a pharmaceutically acceptable salt thereof, or a pharmaceutical composition thereof.

In some embodiments, the present invention provides a method of treating a cancer in a patient, comprising measuring staining intensity in a tumor tissue section of a patient using an EphA2 IHC staining assay, selecting a patient having an H-score for tumor cell membrane of about 15 or more, about 20 or more, about 30 or more, about 40 or more, about 50 or more, about 75 or more, about 100 or more, about 125 or more, or about 150 or more, and administering BT5528 to the patient, or a pharmaceutically acceptable salt thereof, or a pharmaceutical composition thereof.

A Bicycle toxin conjugate specific for EphA2, or a pharmaceutically acceptable salt thereof, or a pharmaceutical composition thereof, can be administered to a patient at various dose ranges.

In some embodiments, a method of the present invention comprises administering a Bicycle toxin conjugate specific for EphA2, or a pharmaceutically acceptable salt thereof, or a pharmaceutical composition thereof, to a patient at a dose of about 1 mg/kg or less. In some embodiments, a method of the present invention comprises administering a Bicycle toxin conjugate specific for EphA2, or a pharmaceutically acceptable salt thereof, or a pharmaceutical composition thereof, to a patient at a dose of about 0.9 mg/kg, about 0.8 mg/kg, about 0.7 mg/kg, about 0.6 mg/kg, about 0.5 mg/kg, about 0.4 mg/kg, about 0.3 mg/kg, about 0.2 mg/kg, or about 0.1 mg/kg.

In some embodiments, a method of the present invention comprises administering a Bicycle toxin conjugate specific for EphA2, or a pharmaceutically acceptable salt thereof, or a pharmaceutical composition thereof, to a patient at a dose of about 100 mg/m² or less. In some embodiments, a method of the present invention comprises administering a Bicycle toxin conjugate specific for EphA2, or a pharmaceutically acceptable salt thereof, or a pharmaceutical composition thereof, to a patient at a dose of about 90 mg/m², about 80 mg/m², about 70 mg/m², about 60 mg/m², about 50 mg/m², about 40 mg/m², about 30 mg/m², about 25 mg/m², about 22.5 mg/m², about 20 mg/m², about 17.5 mg/m², about 15 mg/m², about 12.5 mg/m², about 10 mg/m², about 7.5 mg/m², about 5 mg/m², about 2.5 mg/m², or about 1 mg/m². In some embodiments, a method of the present invention comprises administering a Bicycle toxin conjugate specific for EphA2, or a pharmaceutically acceptable salt thereof, or a pharmaceutical composition thereof, to a patient at a dose of about 2 mg/m² to about 25 mg/m².

A Bicycle toxin conjugate specific for EphA2, or a pharmaceutically acceptable salt thereof, or a pharmaceutical composition thereof, can be administered to a patient at various dose frequencies. In some embodiments, a method of the present invention comprises administering a Bicycle toxin conjugate specific for EphA2, or a pharmaceutically acceptable salt thereof, or a pharmaceutical composition thereof, to a patient at a dose frequency of one dose every 2 days, one dose every 3 days, one dose every 4 days, one dose every 5 days, one dose every 6 days, or one dose every 7 days. In some embodiments, a method of the present invention comprises administering a Bicycle toxin conjugate specific for EphA2, or a pharmaceutically acceptable salt thereof, or a pharmaceutical composition thereof, to a patient at a dose frequency of two doses every week, one dose every week, one dose every two weeks, one dose every three weeks, or one dose every 4 weeks.

### 4. Formulation and Administration

In some embodiments, a method described herein comprises administering a pharmaceutical composition comprising a Bicycle toxin conjugate specific for EphA2, or a pharmaceutically acceptable salt thereof, as described herein, and a pharmaceutically acceptable carrier, adjuvant, or vehicle. In some embodiments, a Bicycle toxin conjugate specific for EphA2, or a pharmaceutically acceptable salt thereof, is formulated for IV administration to a patient.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-1 mg/kg body weight/day can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### 5. Uses

In some embodiments, the present invention provides a method of treating a cancer in a patient comprising selecting a patient having an elevated EphA2 level in a tumor tissue, for example, using a method as described herein, and administering to the patient a Bicycle toxin conjugate specific for EphA2, or a pharmaceutically acceptable salt thereof, or a pharmaceutical composition thereof. In some embodiments, a treatment method further comprises measuring the EphA2 level in a tumor tissue of a patient, for example, using an IHC assay as described herein.

In some embodiments, the present invention provides a method of treating a cancer in a patient having an elevated EphA2 level in a tumor tissue, comprising administering to the patient a Bicycle toxin conjugate specific for EphA2, or a pharmaceutically acceptable salt thereof, or a pharmaceutical composition thereof. In some embodiments, a treatment method further comprises measuring the EphA2 level in a tumor tissue of a patient, for example, using an IHC method as described herein.

Cancer

The cancer or proliferative disorder or tumor to be treated using the methods and uses described herein include, but are not limited to, a hematological cancer, a lymphoma, a myeloma, a leukemia, a neurological cancer, skin cancer, breast cancer, a prostate cancer, a colorectal cancer, lung cancer, head and neck cancer, a gastrointestinal cancer, a liver cancer, a pancreatic cancer, a genitourinary cancer, a bone cancer, renal cancer, and a vascular cancer.

A cancer to be treated using the methods described herein can be selected from colorectal cancer, such as microsatellite-stable (MSS) metastatic colorectal cancer, including advanced or progressive microsatellite-stable (MSS) CRC; non-small cell lung cancer (NSCLC), such as advanced and/or metastatic NSCLC; ovarian cancer; breast cancer, such as inflammatory breast cancer; endometrial cancer; cervical cancer; head and neck cancer; gastric cancer; gastroesophageal junction cancer; and bladder cancer. In some embodiments, a cancer is colorectal cancer. In some embodiments, the colorectal cancer is metastatic colorectal cancer. In some embodiments, the colorectal cancer is microsatellite-stable (MSS) metastatic colorectal cancer. In some embodiments, a cancer is advanced or progressive microsatellite-stable (MSS) CRC. In some embodiments, a cancer is non-small cell lung cancer (NSCLC). In some embodiments, a cancer is advanced and/or metastatic NSCLC. In some embodiments, a cancer is ovarian cancer. In some embodiments, a cancer is breast cancer. In some embodiments, a cancer is inflammatory breast cancer. In some embodiments, a cancer is endometrial cancer. In some embodiments, a cancer is cervical cancer. In some embodiments, a cancer is head and neck cancer. In some embodiments, a cancer is gastric cancer. In some embodiments, a cancer is gastroesophageal junction cancer. In some embodiments, a cancer is bladder cancer.

Cancer includes, in some embodiments, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (WA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

Cancer includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, the cancer is Acute Lymphoblastic Leukemia (ALL), Acute Myeloid Leukemia (AML), Adrenocortical Carcinoma, Anal Cancer, Appendix Cancer, Atypical Teratoid/Rhabdoid Tumor, Basal Cell Carcinoma, Bile Duct Cancer, Bladder Cancer, Bone Cancer, Brain Tumor, Astrocytoma, Brain and Spinal Cord Tumor, Brain Stem Glioma, Central Nervous System Atypical Teratoid/Rhabdoid Tumor, Central Nervous System Embryonal Tumors, Breast Cancer, Bronchial Tumors, Burkitt Lymphoma, Carcinoid Tumor, Carcinoma of Unknown Primary, Central Nervous System Cancer, Cervical Cancer, Childhood Cancers, Chordoma, Chronic Lymphocytic Leukemia (CLL), Chronic Myelogenous Leukemia (CIVIL), Chronic Myeloproliferative Disorders, Colon Cancer, Colorectal Cancer, Craniopharyngioma, Cutaneous T-Cell Lymphoma, Ductal Carcinoma In Situ (DCIS), Embryonal Tumors, Endometrial Cancer, Ependymoblastoma, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Fibrous Histiocytoma of Bone, Gallbladder Cancer, Gastric Cancer, Gastrointestinal Carcinoid Tumor, Gastrointestinal Stromal Tumors (GIST), Germ Cell Tumor, Ovarian Germ Cell Tumor, Gestational Trophoblastic Tumor, Glioma, Hairy Cell Leukemia, Head and Neck Cancer, Heart Cancer, Hepatocellular Cancer, Histiocytosis, Langerhans Cell Cancer, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer, Lobular Carcinoma In Situ (LCIS), Lung Cancer, Lymphoma, AIDS-Related Lymphoma, Macroglobulinemia, Male Breast Cancer, Medulloblastoma, Medulloepithelioma, Melanoma, Merkel Cell Carcinoma, Malignant Mesothelioma, Metastatic Squamous Neck Cancer with Occult Primary, Midline Tract Carcinoma Involving NUT Gene, Mouth Cancer, Multiple Endocrine Neoplasia Syndrome, Multiple Myeloma/Plasma Cell Neoplasm, Mycosis Fungoides, Myelodysplastic Syndrome, Myelodysplastic/Myeloproliferative Neoplasm, Chronic Myelogenous Leukemia (CIVIL), Acute Myeloid Leukemia (AML), Myeloma, Multiple Myeloma, Chronic Myeloproliferative Disorder, Nasal Cavity Cancer, Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip Cancer, Oropharyngeal Cancer, Osteosarcoma, Ovarian Cancer, Pancreatic Cancer, Papillomatosis, Paraganglioma, Paranasal Sinus Cancer, Nasal Cavity Cancer, Parathyroid Cancer, Penile Cancer, Pharyngeal Cancer, Pheochromocytoma, Pineal Parenchymal Tumors of Intermediate Differentiation, Pineoblastoma, Pituitary Tumor, Plasma Cell Neoplasm, Pleuropulmonary Blastoma, Breast Cancer, Primary Central Nervous System (CNS) Lymphoma, Prostate Cancer, Rectal Cancer, Renal Cell Cancer, Clear cell renal cell carcinoma, Renal Pelvis Cancer, Ureter Cancer, Transitional Cell Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma, Squamous Neck Cancer with Occult Primary, Squamous Cell Carcinoma of the Head and Neck (HNSCC), Stomach Cancer, Supratentorial Primitive Neuroectodermal Tumors, T-Cell Lymphoma, Testicular Cancer, Throat Cancer, Thymoma, Thymic Carcinoma, Thyroid Cancer, Transitional Cell Cancer of the Renal Pelvis and Ureter, Triple Negative Breast Cancer (TNBC), Gestational Trophoblastic Tumor, Unknown Primary, Unusual Cancer of Childhood, Urethral Cancer, Uterine Cancer, Uterine Sarcoma, Waldenstrom Macroglobulinemia, or Wilms Tumor.

In certain embodiments, the cancer is selected from bladder cancer, breast cancer (including TNBC), cervical cancer, colorectal cancer, chronic lymphocytic leukemia (CLL), diffuse large B-cell lymphoma (DLBCL), esophageal adenocarcinoma, glioblastoma, head and neck cancer, leukemia (acute and chronic), low-grade glioma, lung cancer (including adenocarcinoma, non-small cell lung cancer, and squamous cell carcinoma), Hodgkin's lymphoma, non-Hodgkin lymphoma (NHL), melanoma, multiple myeloma (MM), ovarian cancer, pancreatic cancer, prostate cancer, renal cancer (including renal clear cell carcinoma and kidney papillary cell carcinoma), and stomach cancer.

In some embodiments, the cancer is small cell lung cancer, non-small cell lung cancer, colorectal cancer, multiple myeloma, acute myeloid leukemia (AML), acute lymphoblastic leukemia (ALL), pancreatic cancer, liver cancer, hepatocellular cancer, neuroblastoma, other solid tumors or other hematological cancers.

In some embodiments, the cancer is small cell lung cancer, non-small cell lung cancer, colorectal cancer, multiple myeloma, or AML.

The present invention further features methods and compositions for the diagnosis, prognosis and treatment of viral-associated cancers, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+T-cell leukemia characterized by clonal integration of HTLV-I in leukemic cells (See https://clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See https://clinicaltrials.gov/ct2/show/study/NCT02488759; see also https://clinicaltrials.gov/ct2/show/study/NCT0240886; https://clinicaltrials.gov/ct2/show/NCT02426892)

In some embodiments, the cancer or tumor comprises any of the cancers described herein. In some embodiments, the cancer comprises melanoma cancer. In some embodiments, the cancer comprises breast cancer. In some embodiments, the cancer comprises lung cancer. In some embodiments the cancer comprises small cell lung cancer (SCLC). In some embodiments, the cancer comprises non-small cell lung cancer (NSCLC).

In some embodiments, the methods or uses described herein inhibit or reduce or arrest the growth or spread of a cancer or tumor. In some embodiments, the methods or uses described herein inhibit or reduce or arrest further growth of the cancer or tumor. In some embodiments, the methods or uses described herein reduce the size (e.g., volume or mass) of the cancer or tumor by at least 5%, at least 10%, at least 25%, at least 50%, at least 75%, at least 90% or at least 99% relative to the size of the cancer or tumor prior to treatment. In some embodiments, the methods or uses described herein reduce the quantity of the cancers or tumors in the patient by at least 5%, at least 10%, at least 25%, at least 50%, at least 75%, at least 90% or at least 99% relative to the quantity of cancers or tumors prior to treatment.

The compounds and compositions, according to the methods of the present invention, can be administered using any amount and any route of administration effective for treating or lessening the severity of a cancer or tumor. The exact amount required varies from subject to subject, depending on the species, age, and general condition of the subject, the severity of the disease or condition, the particular agent, its mode of administration, and the like. The compounds and compositions, according to the methods of the present invention, are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions is decided by the attending physician within the scope of sound medical judgment. The specific effective dose level for any particular patient or organism depends upon a variety of factors, including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The terms “patient” or “subject,” as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the disease or disorder being treated. In certain embodiments, the compounds of the invention can be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound as described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type can also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### EXEMPLIFICATION

The following examples are intended to illustrate the invention and are not to be construed as being limitations thereon. All amino acids, unless noted otherwise, were used in the L-configurations.

| Abbreviations | Name |
|---|---|
| Ac | Acetyl |
| β-Ala | P-Alanine |
| D-Asp | D-Aspartic acid |
| HArg | HomoArginine |
| HyP | Hydroxyproline |
| Sar | Sarcosine, such that Sarₓ represents x Sar residues |

### Example 1: Synthesis of BT5528

Preparation of Bicycle Peptide 1

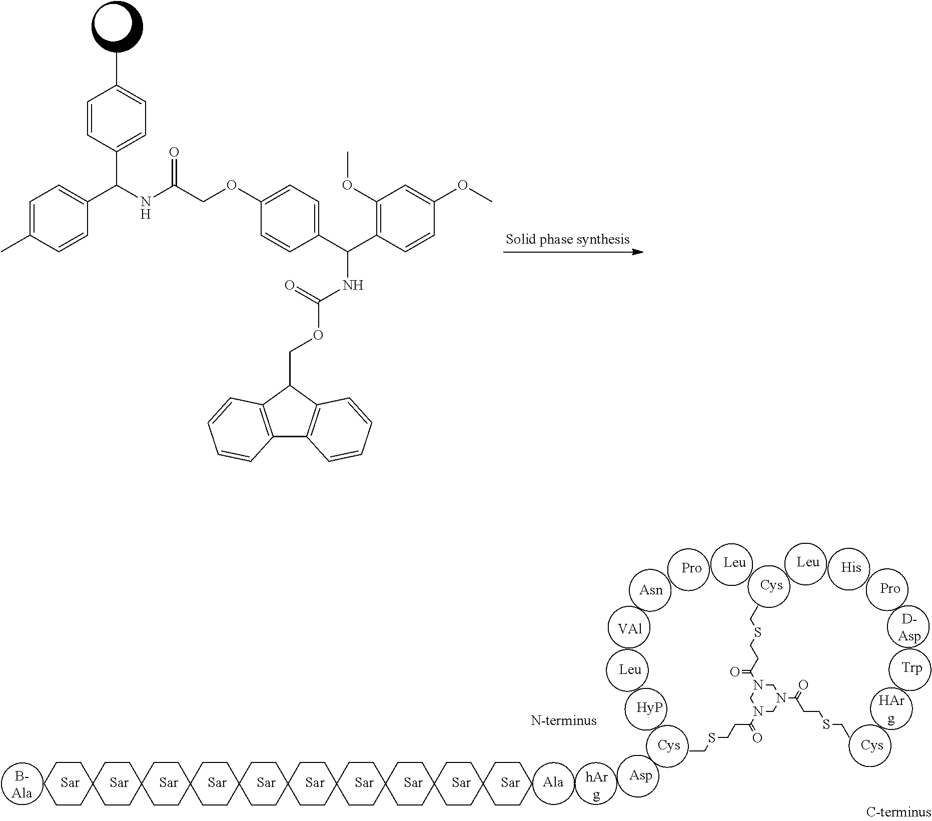

Peptides were synthesized by solid phase synthesis. Rink Amide MBHA Resin was used. To a mixture containing Rink Amide MBHA (0.4-0.45 mmol/g) and Fmoc-Cys(Trt)-OH (3.0 eq) was added DMF, then DIC (3 eq) and HOAt (3 eq) were added and mixed for 1 hour. 20% piperidine in DMF was used for deblocking. Each subsequent amino acid was coupled with 3 eq using activator reagents, DIC (3.0 eq) and HOAT (3.0 eq) in DMF. The reaction was monitored by ninhydrin color reaction or tetrachlor color reaction. After synthesis completion, the peptide resin was washed with DMF×3, MeOH×3, and then dried under N₂ bubbling overnight. The peptide resin was then treated with 92.5% TFA/2.5% TIS/2.5% EDT/2.5% H₂O for 3 h. The peptide was precipitated with cold isopropyl ether and centrifuged (3 min at 3000 rpm). The pellet was washed twice with isopropyl ether and the crude peptide was dried under vacuum for 2 hours and then lyophilised. The lyophilised powder was dissolved in of ACN/H₂O (50:50), and a solution of 100 mM TATA in ACN was added, followed by ammonium bicarbonate in H₂O (1M) and the solution mixed for 1 h. Once the cyclisation was complete, the reaction was quenched with 1M aq. Cysteine hydrochloride (10 eq relative to TATA), then mixed and left to stand for an hour. The solution was lyophilised to afford crude product. The crude peptide was purified by Preparative HPLC and lyophilized to give Bicycle Peptide 1, having amino acid Sequence: (β-Ala)-Sar₁₀-(SEQ ID NO: 1)—CONH₂.

8.0 g of resin was used to generate 2.1 g Bicycle Peptide 1 (99.2% purity; 16.3% yield) as a white solid.

Bicycle Peptide 1 Analytical Data

| Mobile Phase: | A: 0.1% TFA in H2O B: 0.1% TFA in ACN |
|---|---|
| Flow: | 1.0 ml/min |
| Column: | Gemini-NX C18 5 um 110A 150*4.6 mm |
| Instrument: | Agilent 1200 HPLC-BE(1-614) |
| Method: | 15-45% B over 20 minutes, then 3 min 95% B |
| Retention Time: | 11.31 min |
| LCMS (ESI): | m/z 1061.8 [M + 3H]³⁺, 796.5 [M + 4H]⁴⁺ |
| Peptide mw | 3183.68 |

Preparation of MMAE-PABC-Cit-Val-Glutarate-NHS

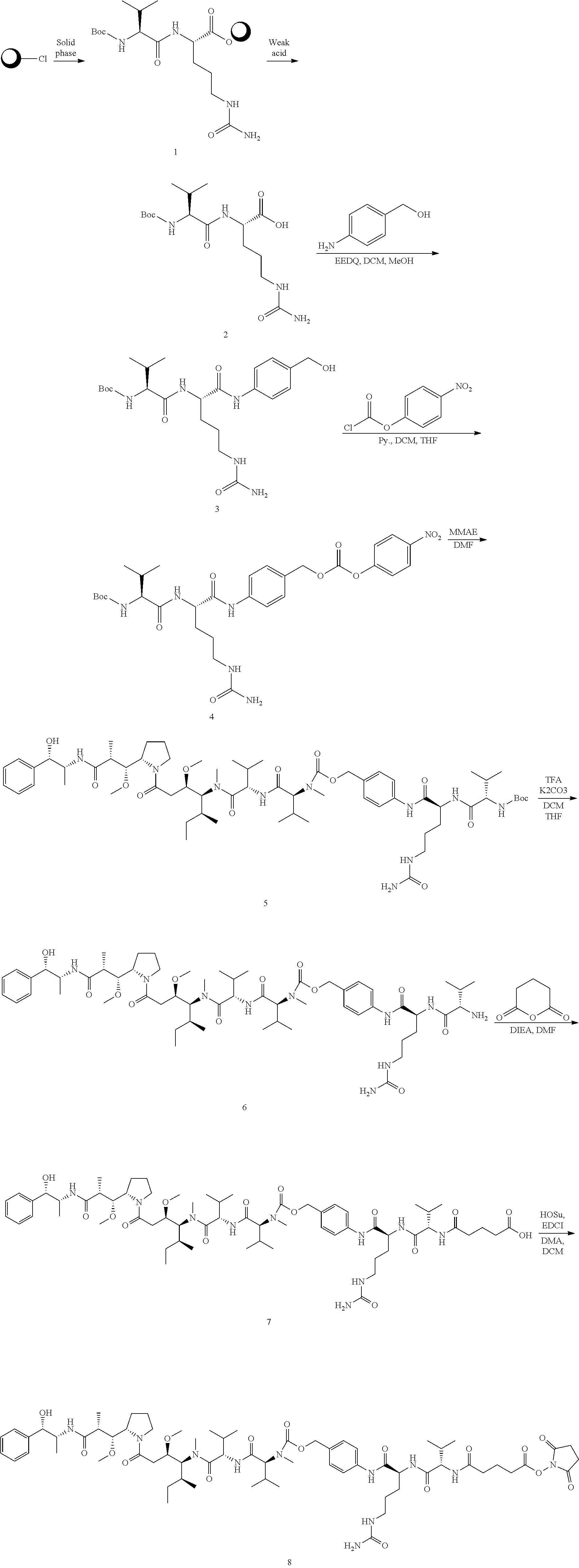

Preparation of Compound 2

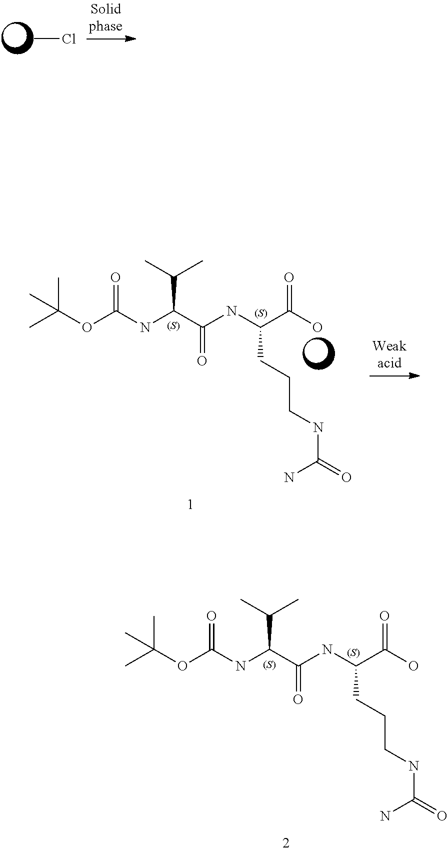

The peptide was synthesized by solid phase synthesis. 50 g CTC Resin (sub: 1.0 mmol/g) was used. To a mixture containing CTC Resin (50 mmol, 50 g, 1.0 mmol/g) and Fmoc-Cit-OH (19.8 g, 50 mmol, 1.0 eq) was added DCM (400 mL), then DIEA (6.00 eq) was added and mixed for 3 hours. And then MeOH (50 mL) was added and mixed for 30 min for capping. 20% piperidine in DMF was used for deblocking. Boc-Val-OH (32.5 g, 150 mmol, 3 eq) was coupled with 3 eq using HBTU (2.85 eq) and DIPEA (6.0 eq) in DMF (400 mL). The reaction was monitored by ninhydrin colour reaction test. After synthesis completion, the peptide resin was washed with DMF×3, MeOH×3, and then dried under N₂ bubbling overnight. After that the peptide resin was treated with 20% HFIP/DCM for 30 min for 2 times. The solution was removed on a rotary evaporator to give the crude. The crude peptide was dissolved in ACN/H2O, then lyophilized twice to give the peptide product (17.3 g crude).

| LCMS (ESI): | m/z 374.9 [M + H]⁺ |
|---|---|
| Molecular weight | 374.44 |

Preparation of Compound 3

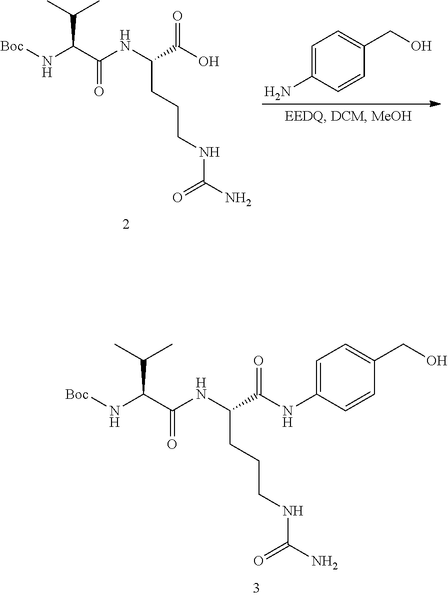 [SMILES: CC(C)[C@H](NC(=O)OC(C)(C)C)C(=O)N[C@@H](CCCNC(N)=O)C(=O)O ; Nc1ccc(CO)cc1 ; CC(C)[C@H](NC(=O)OC(C)(C)C)C(=O)N[C@@H](CCCNC(N)=O)C(=O)Nc1ccc(CO)cc1]

A solution of Compound 2 (4.00 g, 10.68 mmol, 1.00 eq) in DCM (40.00 mL) and MeOH (20.00 mL) was stirred at room temperature, then (4-aminophenyl)methanol (1.58 g, 12.82 mmol, 1.20 eq) and EEDQ (5.28 g, 21.37 mmol, 2.00 eq) were added and the mixture stirred in the dark for 9 hrs. TLC (dichloromethane/methanol=5/1, Rf=0.56) indicated one new spot had formed. The reaction mixture was concentrated under reduced pressure to remove solvent. The resulting residue was purified by flash silica gel chromatography (ISCO®; 120 g SepaFlash® Silica Flash Column, Eluent of 0˜20% MeOH/DCM @ 80 mL/min). Compound 3 (3.00 g, 6.26 mmol, 58.57% yield) was obtained as a white solid.

| LCMS (ESI): | m/z 480.1 [M + H]⁺ |
|---|---|
| Molecular weight | 479.58 |

Preparation of Compound 4

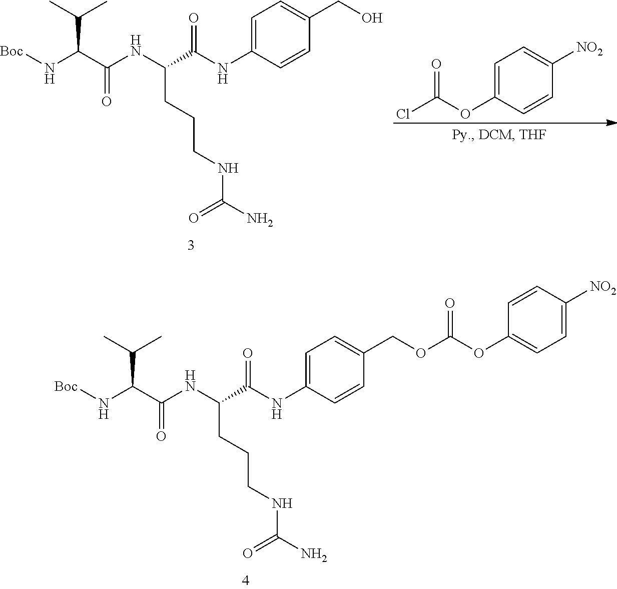 [SMILES: CC(C)[C@H](NC(=O)OC(C)(C)C)C(=O)N[C@@H](CCCNC(N)=O)C(=O)Nc1ccc(CO)cc1 ; O=C(Cl)Oc1ccc([N+](=O)[O-])cc1 ; CC(C)[C@H](NC(=O)OC(C)(C)C)C(=O)N[C@@H](CCCNC(N)=O)C(=O)Nc1ccc(COC(=O)Oc2ccc([N+](=O)[O-])cc2)cc1]

To a solution of Compound 3 (3.00 g, 6.26 mmol, 1.00 eq) in anhydrous THF (35.00 mL) and anhydrous DCM (15.00 mL) was added (4-nitrophenyl) chloroformate (6.31 g, 31.30 mmol, 5.00 eq) and pyridine (2.48 g, 31.30 mmol, 2.53 mL, 5.00 eq), and the mixture was stirred at 25° C. for 5 hrs. TLC (dichloromethane/methanol=10/1, Rf=0.55) indicated a new spot had formed. The reaction mixture was filtered, and the filtrate was concentrated under reduced pressure to give a residue. The residue was purified by flash silica gel chromatography (ISCO®; 120 g SepaFlash® Silica Flash Column, Eluent of 0˜10% DCM/MeOH@80 mL/min). Compound 4 (2.00 g, 3.10 mmol, 49.56% yield) was obtained as a white solid.

| LCMS (ESI): | m/z 667.3 [M + Na]⁺ |
|---|---|
| Molecular weight | 644.68 |

Preparation of Compound 5

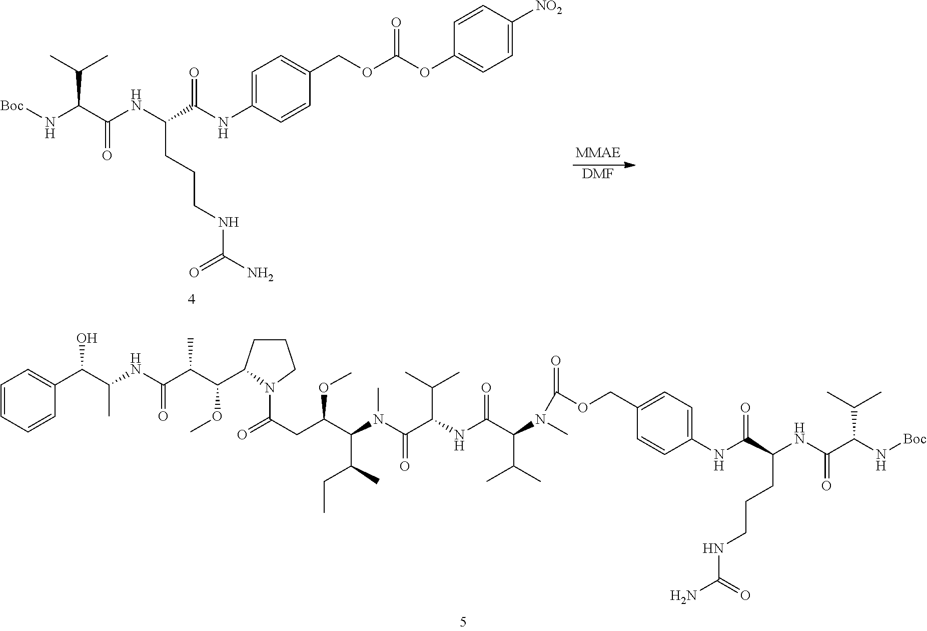

A mixture of Compound 4 (278.43 mg, 387.80 μmol, 1.00 eq) and DIEA (501.19 mg, 3.88 mmol, 677.29 μL, 10.00 eq) in DMF (5.00 mL) was stirred under nitrogen for 10 min. MMAE (250.00 mg, 387.80 μmol, 1.00 eq) and HOBt (52.40 mg, 387.80 μmol, 1.00 eq) were added and the mixture was stirred at 0° C. under nitrogen for 20 min and stirred at 30° C. for additional 18 hrs. LC-MS showed one main peak with desired mass was detected. The resulting mixture was purified by flash C18 gel chromatography (ISCO®; 130 g SepaFlash® C18 Flash Column, Eluent of 0˜50% MeCN/H₂O @ 75 mL/min). Compound 5 (190.00 mg, 155.29 μmol, 40.04% yield) was obtained as a white solid.

| LCMS (ESI): | m/z 1223.4 [M + H]⁺ |
|---|---|
| Molecular weight | 1223.57 |

Preparation of Compound 6

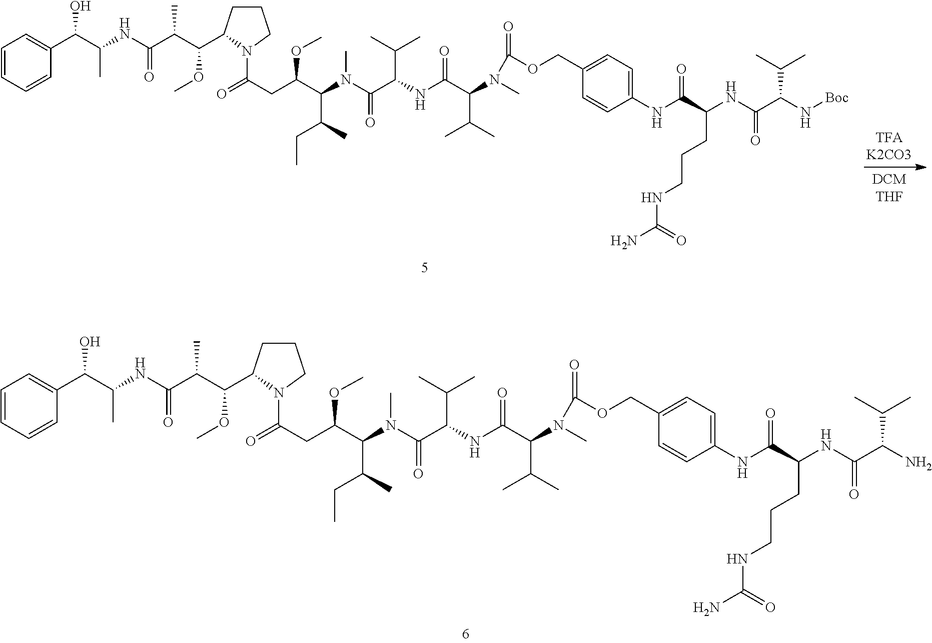 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)OC(C)(C)C)C(C)C)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C]

To a solution of Compound 5 (170.00 mg, 138.94 μmol, 1.00 eq) in DCM (2.70 mL) was added 2,2,2-trifluoroacetic acid (413.32 mg, 3.62 mmol, 268.39 μL, 26.09 eq), and the mixture was stirred at 25° C. for 1 hr. LC-MS showed Compound 5 was consumed completely. The mixture was concentrated under reduced pressure to give a residue. The residue was dissolved in THF (10.00 mL) and was added K₂CO3 (192.03 mg, 1.39 mmol, 10.00 eq), the mixture was stirred at room temperature for additional 3 hrs. LC-MS showed one main peak with desired mass was detected. The resulting reaction mixture was concentrated under reduced pressure to remove solvent to give a residue. The residue was purified by flash C18 gel chromatography (ISCO®; 130 g SepaFlash® C18 Flash Column, Eluent of 0˜50% MeCN/H₂O @ 75 mL/min). Compound 6 (110.00 mg, 97.92 μmol, 70.48% yield) was obtained as a white solid.

| LCMS (ESI): | m/z 1123.4 [M + H]⁺ |
|---|---|
| Molecular weight | 1123.45 |

Preparation of Compound 7

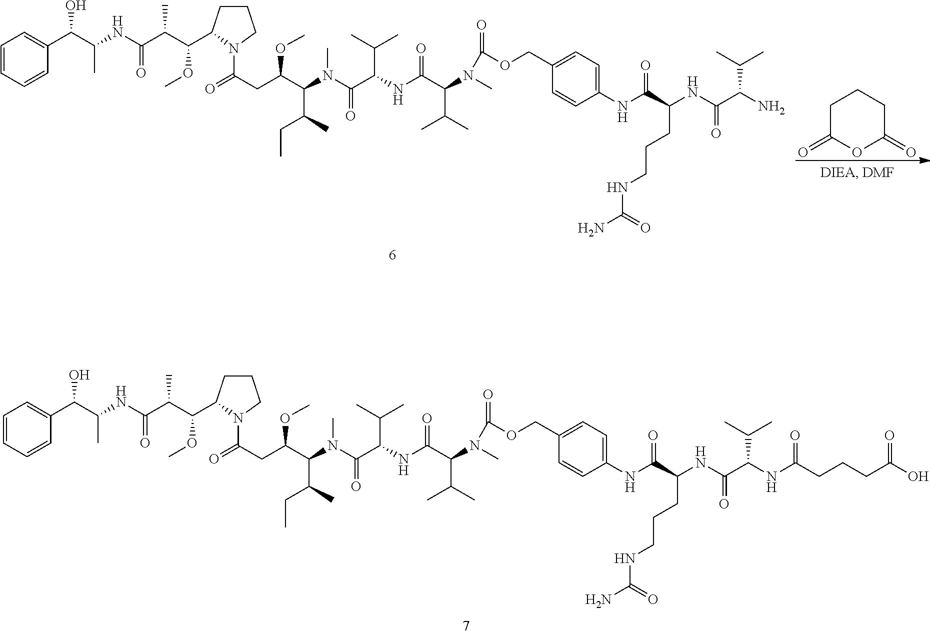 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C ; O=C1CCCC(=O)O1 ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCC(=O)O)C(C)C)cc1)C(C)C]

To a solution of Compound 6 (110.00 mg, 97.92 μmol, 1.00 eq) in DMA (5 mL), DIEA (25.31 mg, 195.83 μmol, 34.20 μL, 2.00 eq) and tetrahydropyran-2,6-dione (22.34 mg, 195.83 μmol, 2.00 eq). The mixture was stirred at room temperature for 18 hrs. LC-MS showed Compound 6 was consumed completely and one main peak with desired mass was detected. The reaction mixture was purified by flash C18 gel chromatography (ISCO®; 130 g SepaFlash® C18 Flash Column, Eluent of 0˜50% MeCN/H₂O @ 75 mL/min). Compound 7 (100.00 mg, 80.81 μmol, 82.53% yield) was obtained as a white solid.

| LCMS (ESI): | m/z 1237.4 [M + H]⁺ |
|---|---|
| Molecular weight | 1236.74 |

Preparation of Compound 8 (MMAE-PABC-Cit-Val-Glutarate-NHS)

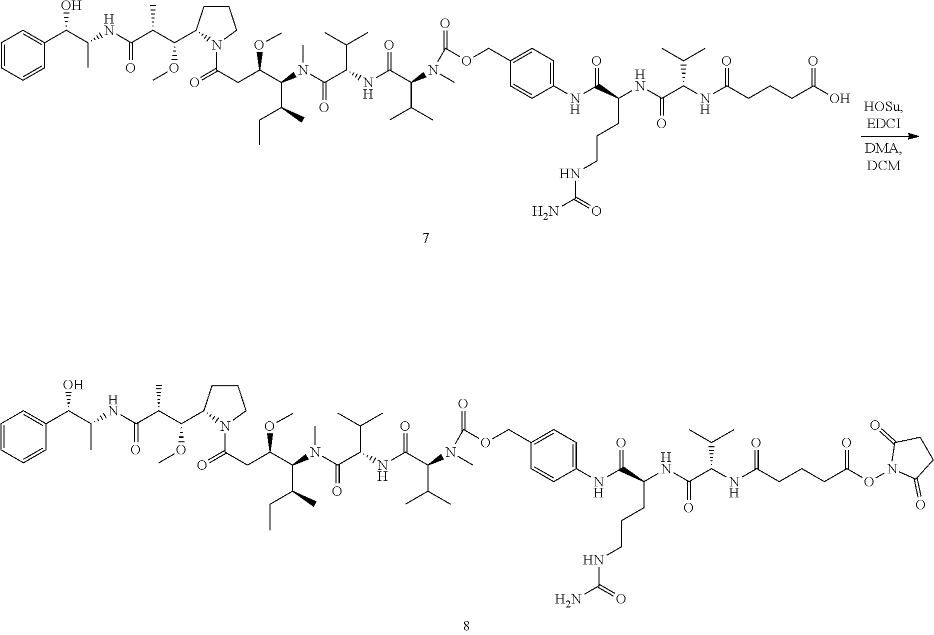 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCC(=O)O)C(C)C)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCC(=O)ON2C(=O)CCC2=O)C(C)C)cc1)C(C)C]

To a solution of Compound 7 (100.00 mg, 80.81 μmol, 1.00 eq) in DMA (4.5 mL) and DCM (1.5 mL) was added 1-hydroxypyrrolidine-2,5-dione (27.90 mg, 242.42 μmol, 3.00 eq) under N₂, the mixture was stirred at 0° C. for 30 min. EDCI (46.47 mg, 242.43 μmol, 3.00 eq) was added in the mixture, and the mixture was stirred at 25° C. for additional 16 hrs. LC-MS showed Compound 7 was consumed completely and one main peak with desired mass was detected. The reaction mixture was purified by flash C18 gel chromatography (ISCO®; 130 g SepaFlash® C18 Flash Column, Eluent of 0˜50% MeCN/H₂O @ 75 mL/min). Compound 8 (90.00 mg, 60.69 μmol, 75.11% yield) was obtained as a white solid.

| LCMS (ESI): | m/z 1334.5 [M + H]⁺ |
|---|---|
| Molecular weight | 1334.62 |

Preparation of BT5528

To a solution of Bicycle Peptide 1 (1.0-1.3 eq) in DMA was added DIEA (3 eq) and compound 8 (1 eq). The mixture was stirred at 25° C. for 18 hr. The reaction was monitored by LC-MS and once complete, was directly purified by preparative HPLC.

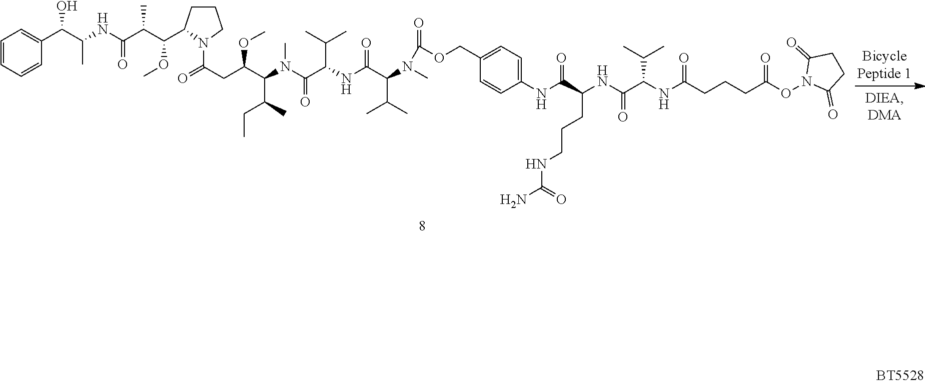 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCC(=O)ON2C(=O)CCC2=O)C(C)C)cc1)C(C)C]

Bicycle Peptide 1 (71.5 mg, 22.48 μmol) was used as the bicycle reagent. BT5528 (40.9 mg, 9.05 μmol, 40.27% yield, 97.42% purity) was obtained as a white solid.

BT5528 Analytical Data

| Mobile Phase: | A: 0.1% TFA in H2O B: 0.1% TFA in ACN |
|---|---|
| Flow: | 1.0 ml/min |
| Column: | Gemini-NX C18 5 um 110A 150*4.6 mm |
| Instrument: | Agilent 1200 HPLC-BE(1-614) |
| Method: | 28-68% B over 30 minutes, then 3 min 95% B |
| Retention Time: | 11.35 min |
| LCMS (ESI): | m/z 1468.1 [M + 3H]³⁺, 1101.2 [M + 4H]⁴⁺, |
| | 881.3 [M + 5H]⁵⁺ |
| Peptide mw | 4404.2 |

### Example 2. EphA2 IHC Assay

This assay detects the EphA2 extracellular domain (ECD), which is BT5528's binding site.

### 1. Reagents/Probes/Antibodies

| | Storage |
|---|---|
| Name | Temperature |
| Human EphA2 antibody [Polyclonal Goat IgG] (R&D | −20° C. |
| Systems, Cat# AF3035) | |
| Normal Goat IgG Control (R&D Systems, Cat # AB-108-C) | −20° C. |
| Protein Block, Serum-Free (Agilent/Dako, Cat # X0909) | 2-8° C. |
| Target Retrieval Solution, Low pH (Agilent/Dako, Cat # | 2-8° C. |
| K8005) | |
| ImmPRESS ® HRP Anti-Goat IgG (Peroxidase) Polymer | 2-8° C. |
| Detection Kit, made in Horse (Vector Labs, Cat # MP-7405) | |
| EnVision ™ FLEX, High pH, (Link) Kit: | 2-8° C. |
| EnVision FLEX Peroxidase-Blocking Reagent | |
| Envision FLEX Wash Buffer 20X | |
| DAB Substrate Buffer & Chromogen | |
| (Dako, Cat# K8002) | |
| EnVision ™ FLEX Hematoxylin (Dako, Cat# K8008) | Ambient |
| Deionized Water | Ambient |
| 100% Alcohol | Ambient |
| 95% Alcohol | Ambient |
| Xylene | Ambient |

### 2. Equipment

| Name | Model |
|---|---|
| Refrigerator | Thermo Scientific REL3004A22 or equivalent |
| Microtome | Leica RM2235 or equivalent |
| Drying Oven | Biocare 10-180 Aer Desert Chamber or equivalent |
| Automated IHC Stainer | Dako Link 48 |
| Pretreatment Module | Dako PT Link |
| Linear Stainer | Leica Autostainer XL or equivalent |
| Coverslipper | Sakura Tissue-Tek Film 4740 or equivalent |
| Water Bath | TBS TFBL or equivalent |

### 3. Assay Procedures

1. Fix and embed the tissue, cut and mount the sections to positively charged slides, deparaffinize and rehydrate the section per standard practice
2. Load the samples into the Dako PT Link pretreatment module
3. Warm module to 65° C., perform antigen retrieval for 20 minutes at 97° C. with Target Retrieval Solution, Low pH, cool autostainer to 65° C.
4. Unload slides from the PT Link and submerge in Dako wash buffer at room temperature (minimum of 5 minutes)
5. Load the samples into the Dako Autostainer 48
6. Wash with Dako Wash Buffer for 5 min at ambient
7. Block with Flex Peroxide Blocking Reagent for 5 minutes at ambient
8. Rinse with wash buffer
9. Block with Protein Block for 10 minutes at ambient
10. Air blow
11. Incubate with anti-hEphA2 antibody (10 ug/ml) diluted in Dako Background Reducing Diluent for 30 min at ambient
12. Rinse with wash buffer
13. Incubate with normal horse serum, 2.5% for 20 min at ambient
14. Rinse with wash buffer
15. Incubate with Horse Anti-Goat IgG Polymer Reagent for 15 min at ambient
16. Rinse with wash buffer
17. Incubate with FLEX DAB Substrate Buffer & Chromogen for 10 min at ambient
18. Rinse with wash buffer
19. Rinse with DI water
20. Incubate with FLEX Hematoxylin for 1 min at ambient
21. Rinse with DI water
22. Rinse with DI water
23. Unload from Dako, dehydrate in graded alcohols and clear in xylene for 7 min at ambient
24. Coverslip

4. EphA2 IHC Scoring

Score the EphA2 staining results using the H-score method (defined as the sum of the products of the percent of cells x their staining intensity, on a scale of 0-3 where 0 is negative and 3 is strongly stained). Independent H-scores for tumor cell membrane (TM) and tumor cytoplasm (TC) can be generated to differentiate between the two compartments. H-scores≥20 were considered positive for EphA2.

### 5. Results

TMAs of indications including pancreatic, bladder, head & neck, stomach, NSCLC, TNBC, and ovarian cancer were stained and scored for EphA2 expression. The pattern of EphA2 expression was different across indications tested with pancreas having the greatest frequency of EphA2 expression. In contrast to the other indications assessed, pancreas and bladder had a greater percentage of cores positive for TM over TC. These differences may be relevant for BT5528 indication selection given that the mechanism of BTCs may be enhanced in cases which are TM positive.

| | Total | TM Positive | TC Positive | TM or TC Positive |
|---|---|---|---|---|
| Indication | cores (N) | N (%) | N (%) | N (%) |
| Pancreatic | 92 | 48 (52) | 14 (15) | 48 (52) |
| Bladder | 142 | 42 (30) | 37 (26) | 51 (36) |
| Head & Neck | 69 | 14 (20) | 17 (25) | 22 (32) |
| Stomach | 53 | 10 (19) | 15 (28) | 15 (28) |
| NSCLC | 221 | 45 (20) | 52 (24) | 60 (27) |
| Ovarian | 83 | 8 (10) | 13 (16) | 17 (20) |
| TNBC | 124 | 3 (2) | 25 (20) | 25 (20) |

Use of an IHC assay assessing expression of EphA2 across multiple TMAs and scoring TM and TC individually may help guide indication selection for the BT5528 clinical program.

While a number of embodiments of this invention are described, it is apparent that the examples may be altered to provide other embodiments that utilize the compounds and methods of this invention. Therefore, it will be appreciated that the scope of this invention is to be defined by the application and claims rather than by the specific embodiments that have been represented by way of example.

1. A method of identifying or selecting a patient having an elevated EphA2 level in a tumor tissue, comprising measuring EphA2 level in a tumor tissue of a patient, and selecting a patient having an elevated EphA2 level in the tumor tissue.

2. The method of claim 1, wherein the step of measuring EphA2 level in a tumor tissue of a patient comprises using an EphA2 immunohistochemistry (IHC) staining assay.

3. A method of identifying or selecting a patient having an elevated EphA2 level in a tumor tissue, comprising measuring staining intensity in a tumor tissue section of a patient using an EphA2 IHC staining assay, and selecting a patient who is staining positive in the EphA2 IHC staining assay.

4. The method of any one of claims 1-3, wherein the patient has pancreatic cancer, stomach cancer, bladder cancer, head & neck cancer, non-small cell lung cancer (NSCLC), triple negative breast cancer (TNBC), or ovarian cancer.

5. The method of claim 2 or 3, wherein the EphA2 immunohistochemistry (IHC) staining assay uses a human EphA2 antibody selectively binding to the extracellular domain (ECD) or cytoplasm domain of EphA2.

6. The method of claim 5, wherein the human EphA2 antibody selectively binding to the extracellular domain (ECD) of EphA2 is human EphA2 antibody AF3035.

7. The method of claim 3, wherein staining positive in the EphA2 IHC staining assay refers to an H-score of about 15 or more, about 20 or more, about 30 or more, about 40 or more, about 50 or more, about 75 or more, about 100 or more, about 125 or more, or about 150 or more in a tumor tissue section in the EphA2 IHC staining assay.

8. The method of claim 3, wherein staining positive in the EphA2 IHC staining assay refers to an H-score for tumor cell membrane of about 15 or more, about 20 or more, about 30 or more, about 40 or more, about 50 or more, about 75 or more, about 100 or more, about 125 or more, or about 150 or more in a tumor tissue section in the EphA2 IHC staining assay.

9. The method of claim 3, wherein staining positive in the EphA2 IHC staining assay refers to an H-score for tumor cell cytoplasm of about 15 or more, about 20 or more, about 30 or more, about 40 or more, about 50 or more, about 75 or more, about 100 or more, about 125 or more, or about 150 or more in a tumor tissue section in the EphA2 IHC staining assay.

10. A method of treating a cancer in a patient having an elevated EphA2 level in a tumor tissue, comprising administering to the patient a Bicycle toxin conjugate specific for EphA2, or a pharmaceutically acceptable salt thereof, or a pharmaceutical composition thereof.

11. The method of claim 10, wherein a patient having an elevated EphA2 level in a tumor tissue refers to a patient having an H-score of about 15 or more, about 20 or more, about 30 or more, about 40 or more, about 50 or more, about 75 or more, about 100 or more, about 125 or more, or about 150 or more in a tumor tissue section in an EphA2 IHC staining assay.

12. A method of treating a cancer in a patient, comprising selecting a patient having an H-score of about 15 or more, about 20 or more, about 30 or more, about 40 or more, about 50 or more, about 75 or more, about 100 or more, about 125 or more, or about 150 or more in a tumor tissue section in an EphA2 IHC staining assay, and administering to the patient a Bicycle toxin conjugate specific for EphA2, or a pharmaceutically acceptable salt thereof, or a pharmaceutical composition thereof.

13. The method of any one of claims 10-12, wherein a cancer is pancreatic cancer, stomach cancer, bladder cancer, head & neck cancer, non-small cell lung cancer (NSCLC), triple negative breast cancer (TNBC), or ovarian cancer.

14. The method of claim 11 or 12, wherein the EphA2 IHC staining assay uses a human EphA2 antibody selectively binding to the extracellular domain (ECD) or cytoplasm domain of EphA2.

15. The method of claim 14, wherein the human EphA2 antibody selectively binding to the extracellular domain (ECD) of EphA2 is human EphA2 antibody AF3035.

16. The method of claim 12, wherein the H-score refers to an H-score for tumor cell membrane or an H-score for tumor cell cytoplasm.

17. A method of treating a cancer in a patient, comprising selecting a patient having an H-score for tumor cell membrane of about 15 or more, about 20 or more, about 30 or more, about 40 or more, about 50 or more, about 75 or more, about 100 or more, about 125 or more, or about 150 or more in a tumor tissue section in an EphA2 IHC staining assay, and administering to the patient BT5528, or a pharmaceutically acceptable salt thereof, or a pharmaceutical composition thereof.

18. The method of claim 17, wherein a cancer is pancreatic cancer, stomach cancer, bladder cancer, head & neck cancer, non-small cell lung cancer (NSCLC), triple negative breast cancer (TNBC), or ovarian cancer.

19. The method of claim 17, wherein the EphA2 IHC staining assay uses a human EphA2 antibody selectively binding to the extracellular domain (ECD).

20. The method of claim 19, wherein the human EphA2 antibody selectively binding to the extracellular domain (ECD) of EphA2 is human EphA2 antibody AF3035.


SEQUENCE LISTING (1 sequence)

SEQ ID NO: 1 - protein, 18 aa, Artificial
  Synthetic Peptide
  Xaa (2)..(2): Xaa is HArg
  Xaa (5)..(5): Xaa is HyP
  Xaa (15)..(15): Xaa is D-Asp
  Xaa (17)..(17): Xaa is HArg
AXDCXLVNPLCLHPXWXC